(19) United States
(12) Patent Application Publication
(10) Pub. No.: US 2013/0060131 A1
(43) Pub. Date: Mar. 7, 2013

(54) METHOD AND APPARATUS FOR EXAMINING INNER EAR

(71) Applicants: John S. Oghalai, Stanford, CA (US); Brian Edward Applegate, Bryan, TX (US); Simon Shang Gao, Palo Alto, CA (US); Patrick David Raphael, Palo Alto, CA (US)

(72) Inventors: John S. Oghalai, Stanford, CA (US); Brian Edward Applegate, Bryan, TX (US); Simon Shang Gao, Palo Alto, CA (US); Patrick David Raphael, Palo Alto, CA (US)

(73) Assignees: The Texas A&M University System, College Station, TX (US); The Board of Trustees of the Leland Stanford Junior University, Palo Alto, CA (US)

(21) Appl. No.: 13/599,772

(22) Filed: Aug. 30, 2012

Related U.S. Application Data
(60) Provisional application No. 61/530,806, filed on Sep. 2, 2011.

(51) Int. Cl. A61B 1/227; A61B 6/02

(52) U.S. Cl. 600/425

(57) ABSTRACT

An apparatus, for examining an inner ear is provided. An endoscope is provided, comprising a wave guide and an end piece comprising an end window to be placed a first distance from an inner ear, wherein the waveguide focuses light to create a focal plane the first distance from the end window. An optical coherence tomography (OCT) system is connected to a second end of the wave guide and comprises an imaging system connected to the OCT system for generating an image of the inner ear.

### CROSS REFERENCE TO RELATED APPLICATIONS

This application claims priority under 35 U.S.C. §119(e) from co-pending U.S. Provisional Application No. 61/530,806, entitled “TECHNIQUE FOR MEASURING AND ANALYZING SOUND VIBRATIONS WITHIN THE INNER EAR”, filed Sep. 2, 2011, by John S. Oghalai et al. and which is incorporated by reference for all purposes.

### GOVERNMENT RIGHTS

This invention was made with Government support under contract W81XWH-11-2-0004 awarded by the U.S. Army Medical Research and Material Command. The Government has certain rights in this invention.

### BACKGROUND OF THE INVENTION

This invention relates generally to a method and apparatus for examining the inner ear. Human hearing loss often occurs as a result of damage or malformations to the functional soft tissues within the cochlea, but these changes are not appreciable with current medical imaging modalities.

U.S. Pat. No. 8,115,934 to Boppart et al. and issued on Feb. 14, 2012 provides optical coherence tomography (OCT) with an otoscope to the ear drum and middle ear by illuminating the ear drum with an otoscope.

### SUMMARY OF THE INVENTION

In accordance with the invention an apparatus, for examining an inner ear is provided. An endoscope is provided, comprising a wave guide and an end piece comprising an end window to be placed a first distance from an inner ear, wherein the waveguide focuses light to create a focal plane the first distance from the end window. An optical coherence tomography (OCT) system is connected to a second end of the wave guide and comprises an imaging system connected to the OCT system for generating an image of the inner ear.

In another manifestation of the invention, a computer implemented method for examining the inner ear is provided. Spectral domain optical coherence tomography is performed on the inner ear, comprising providing a light beam to the inner ear, receiving light reflected from the inner ear, and generating an image of the inner ear from the received light using an optical coherence tomography (OCT) system.

The invention and objects and features thereof will be more readily apparent from the following detailed description and appended claims when taken with the drawings.

### BRIEF DESCRIPTION OF THE DRAWINGS

FIG. 1A is an illustration of a cochlea.

FIG. 1B illustrates the soft tissue of the cochlea.

FIG. 2A demonstrates a CT image of a cochlea in a deaf child that was read as normal.

FIG. 2B shows a CT image of a grossly malformed cochlea in another deaf child in which there is a complete lack of bone within the modiolus of the cochlea.

FIG. 2C shows an MRI image of a cochlea in a deaf child that was also read as normal.

FIG. 2D shows a cochlea that is about half the size of a normal cochlea.

FIG. 3 is a schematic illustration of an embodiment of the invention that uses a spectral domain OCT system.

FIG. 4A is an OCT image from a P15 mouse cochlea.

FIG. 4B is a magnitude and depth plot of the A-line highlighted in FIG. 4A.

FIG. 5A is a spectral domain OCT image of an adult mouse organ of Corti (unaveraged) as viewed with the apical otic capsule removed.

FIG. 5B is a paraffin-embedded histological section from a P15 mouse cochlea shown for comparison.

FIGS. 6A-F shows representative OCT images of the cochlea.

FIG. 7A-C show unaltered, representative images of the cochlea.

FIG. 7D is a graph of the amount of tissue imaged.

FIG. 7E is a graph of the average signal intensity in different regions.

FIG. 7F is a graph of the contrast percentage of regions.

FIGS. 8A-F show the average intensity OCT images from five slices from the cochlea.

FIG. 9 is a graph of an interleaving example.

FIGS. 10A-B show the results for input frequencies of 3-31 kHz with 0.5 kHz

FIG. 11A shows a cross-sectional image (B_{scan}) through the tympanic membrane.

FIGS. 11B-C show two examples of measured vibrational frequency spectra.

FIG. 12 is a schematic illustration of another embodiment of the invention that uses a spectral domain OCT system with an endoscope and a sound system.

FIG. 13 is a high level block diagram showing a computer system, which is suitable for implementing a central computer used in embodiments of the present invention.

FIG. 14 is a more detailed schematic view of an endoscope.

FIG. 15 is a high level flow chart for a method of examining an inner ear using an embodiment of the invention, which comprises an endoscope.

FIG. 16 is a schematic view of an endoscope in another embodiment of the invention.

FIG. 17 is a schematic illustration of the endoscope of FIG. 16 in an ear.

FIG. 18 is a schematic view of an endoscope in another embodiment of the invention.

FIG. 19 is a schematic illustration of the endoscope of FIG. 18 in an ear.

### DETAILED DESCRIPTION OF ILLUSTRATED EMBODIMENTS

The present invention will now be described in detail with reference to a few preferred embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention.

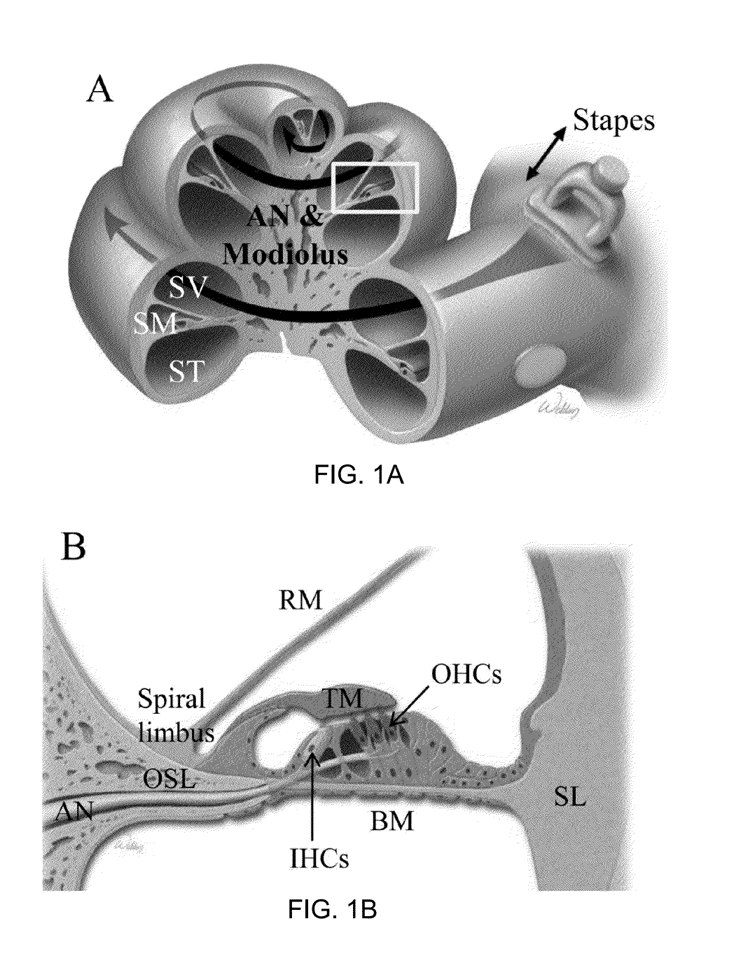

The auditory system serves to amplify and convert sound pressure waves into neuronal signals. Sounds waves are first collected and funneled by the external ear to the tympanic membrane. In mammals, these vibrations are transferred from the external ear through the tympanic membrane (ear drum), to the middle ear comprising of ossicles, and then to the inner ear. The inner ear comprises a cochlea and vestibular system. The cochlea is shown in FIG. 1A. It is spiral shaped and encased in bone, but the key intra-cochlear structures that convert mechanical motion to electrical signals are composed of soft tissue, which is shown in FIG. 1B. More specifically, the inner hair cells (IHCs) and outer hair cells (OHCs) sit atop supporting cells and the basilar membrane (BM), and perform mechano-electrical transduction. The stereocilia of the OHCs connect to the tectorial membrane (TM), which is in turn connected to the spiral limbus. The IHCs relay the afferent signals to the brain via auditory neurons (AN), which are housed in the central, bony modiolus. Reissner's membrane (RM) serves as a diffusion barrier to separate the fluids within scala media (SM) from that of scala vestibuli (SV). The spiral ligament (SL) contains the stria vascularis that maintains the ionic gradients in the SM necessary for normal hearing. Damage to any of these structures results in sensorineural hearing loss. The other half of the inner ear, the vestibular system, functions in a similar manner to convert head movements and gravity sensations to neural signals. Damage to the vestibular system leads to vertigo and/or disequilibrium.

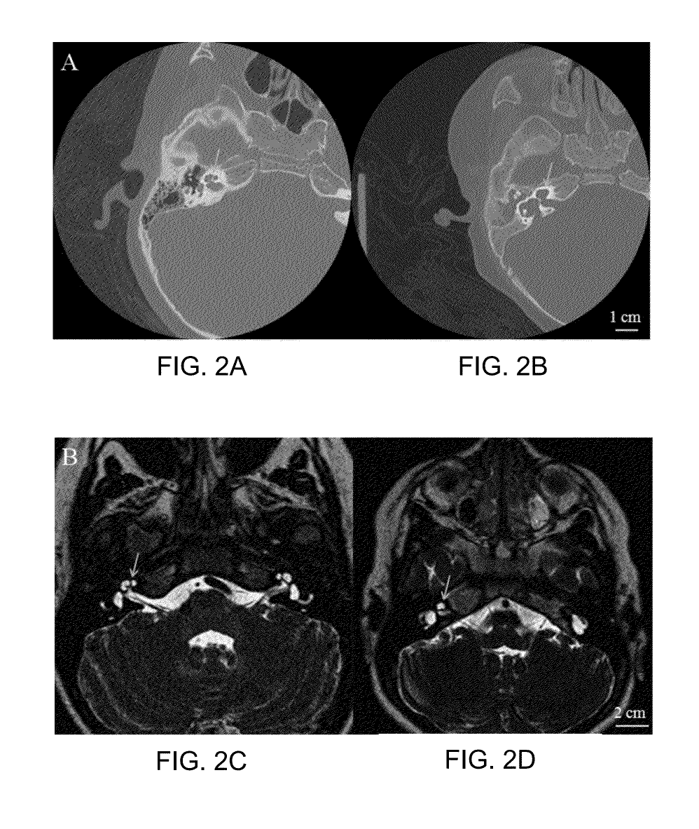

In a human, the cochlea is about 1 cm in diameter, yet the soft tissues range on the order of 10 to 100 μm in thickness. As such, current clinical imaging modalities such as magnetic resonance imaging (MRI) and computed tomography (CT), which have resolutions of approximately 1 and 0.5 mm respectively, simply do not provide the necessary resolution required to detect disturbances in the intra-cochlear soft tissues associated with hearing loss. This is illustrated in FIGS. 2A-D. FIG. 2A demonstrates a CT image of a cochlea (arrow) in a deaf child that was read as normal. Although audiometric tests revealed that the cochlea was malfunctioning, the physical basis for the hearing loss was presumably too small to be appreciated by the imaging technique. CT can, however, detect gross malformations. For example, FIG. 2B shows a CT image of a grossly malformed cochlea (arrow) in another deaf child in which there is a complete lack of bone within the modiolus of the cochlea. Similar problems exist with MRI. FIG. 2C shows an MRI image of a cochlea in a deaf child that was also read as normal, whereas FIG. 2D shows a cochlea that is about half the size of a normal cochlea (arrow).

As seen, current clinical imaging methodologies only allow for the detection of gross bony malformations. However, post-mortem histological analyses of human temporal bones reveal that the most common causes of hearing loss, i.e. age-related, noise-induced, ototoxic exposure, and genetic mutations, only produce changes in the intra-cochlear soft tissues. These changes can include hair cell loss, TM malformation or separation from the OHCs, loss of AN, atrophy of the stria vascularis, and/or loss of auditory neurons. Since most forms of hearing loss do not have any appreciable findings on CT or MRI, this dramatically limits the ability to understand and treat hearing loss in individual patients. The same is true for vertigo and disequilibrium. There are no current imaging modalities to assess the vestibular system within the inner ear.

There is a need for better inner ear imaging technology to help clinicians and researchers visualize the cochlea and vestibular system at a higher resolution. Therefore, an embodiment of the invention applies optical coherence tomography (OCT) to this problem. OCT is a noninvasive imaging technique with micron scale resolution that allows for 3-dimensional imaging within scattering media. An embodiment of the invention uses spectral (or Fourier) domain OCT as it provides a higher signal-to-noise ratio and faster imaging speeds compared to time domain OCT. This may also include Optical Frequency Domain Imaging (OFDI) and Swept Source OCT (SS-OCT).

### System in an Embodiment of the Invention

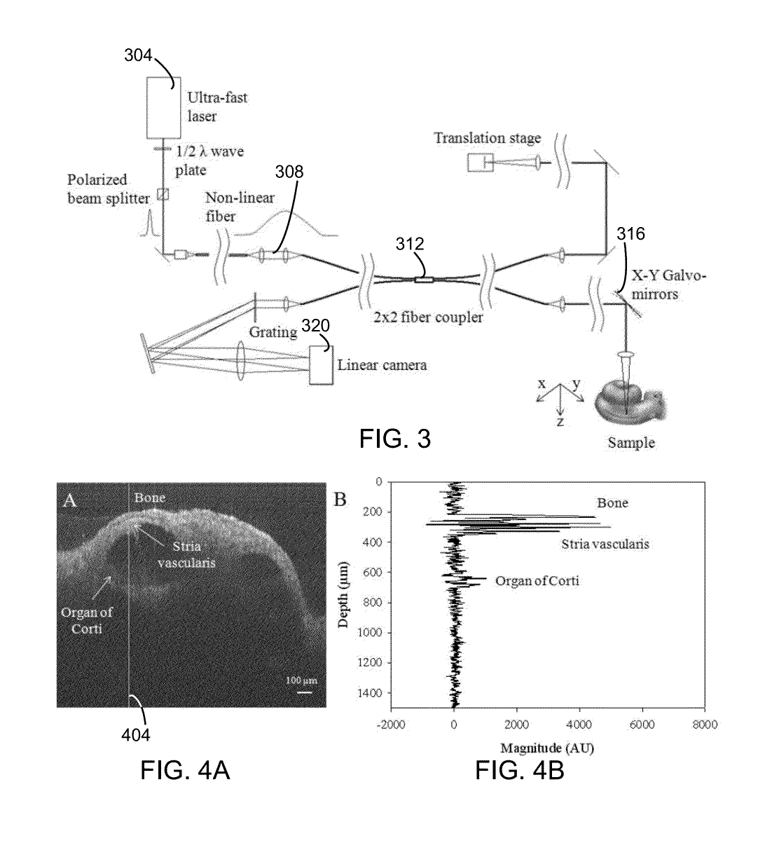

FIG. 3 is a schematic illustration of an embodiment of the invention that uses a spectral domain OCT system. The source 304 consisted of 140 fₛ pulses of 950 nm light from a mode-locked Ti:sapphire laser (Chameleon, Coherent, Santa Clara, Calif.). The light was focused into an ultrahigh numerical aperture single mode optical fiber 308 (UHNA3, NuFern, East Granby, Conn.) in order to broaden the spectral bandwidth. Launching 250 mW into the fiber resulted in spectral broadening of the source to a full width at half maximum of ˜80 nm, resulting in a theoretical axial resolution of ˜5 μm in air or ˜4 μm in solution. The light exiting the fiber was then collimated and focused into a 2×2 (50:50) fiber-fused coupler 312 (WA10500202B2111-BC1, AC Photonics, Santa Clara, Calif.). One of the output ports was coupled into the X-Y galvo-mirror scan head 316 of an upright microscope (MOM, Sutter Instruments, Novato, Calif.), which served as the sample arm; the other was used as the reference arm. The average power incident on the sample tissue surface was ˜10 mW.

The reflected light from both arms was then combined in the fiber-fused coupler 312. The resulting spectral interferogram was measured using a custom spectrometer based on a high speed line scan camera 320 (AViiVA SM2 CL 2014, E2V, Tarrytown, N.Y.) capable of line rates up to 28 kHz. A camera integration time of 30 μs was used for all images presented herein. The dynamic range of the 12-bit camera was ˜70 dB, as referenced to the standard deviation of the dark current and read noise. In custom software written in MATLAB (MathWorks, Natick, Mass.), the interferogram was transformed into k-space, and the magnitude of the Fourier transform was computed to produce the depth-resolved sample reflectivity or A-line. The signal-to-noise ratio of the system was ˜90 dB, as determined by comparing the A-line peak of a mirrored surface to the standard deviation of a region 500 μm away. Three-dimensional images were created from a series of X-Z slices scanned in the Y direction spaced 5 μm apart, each of which was averaged 4 times unless stated otherwise. The lateral resolution, determined experimentally by imaging microspheres, was ˜10 μm. The contrast and intensity curve properties were adjusted in ImageJ or Photoshop CS4 (Adobe, San Jose, Calif.) to optimize the image. However, measurements were made on unaltered images.

### Specimen Preparation for an Embodiment of the Invention

The Stanford University and Baylor College of Medicine Institutional Animal Care and Use Committees approved the study protocols. After sacrifice with an overdose of a ketamine/xylazine mixture, cochleae were isolated from post-natal day 3 (P3), P15, or >P30 (adult) mice. We studied normal-hearing mice (CBA strain) and three genotypes of a transgenic mouse strain that contained a human hearing loss mutation that produces a malformed TM (Tecta+/+(wild-type), Tecta+/C1509G (heterozygous), and TectaC1509G/C1509G (homozygous) genotypes). Each cochlea was glued upright into a chamber before being imaged. The cochlea was immersed in either an external solution of (in mM) 150 NaCl, 4 KCl, 2 MgCl2, 1.5 CaCl2, 10 N-2-hydroxyethylpiperazine-N′-2-ethanesulfonic acid (HEPES), and 10 glucose or phosphate buffered solution (PBS). When indicated in the text, a hole was made in the bone overlaying the region of interest with a fine knife and pick. All images were collected within two hours of animal sacrifice.

### Paraffin-Embedded Histological Sections

Mice were euthanized as previously mentioned. The cochleae were isolated from the temporal bone in PBS and fixed in 4% paraformaldehyde or a solution containing 60% ethanol, 30% formaldehyde, and 10% glacial acetic acid overnight at 4° C. After a triple wash in PBS, the cochleae were decalcified with 0.5 mM ethylenediaminetetraacetic acid (pH 8.0) for 2 days at room temperature. After another set of PBS washes, they were dehydrated with gradient ethanol and Histo-clear (Electron Microscopy Sciences, Hatfield, Pa.) and embedded in paraffin. Serial sections of 7 μm thickness were prepared in the mid-modiolar plane and stained with hematoxylin and eosin. Images were taken at either 5× or 10× magnification on a LSM 5 Exciter (Carl Zeiss, Thornwood, N.Y.).

### Image Analysis

Measurements were made in ImageJ or Photoshop CS4. No adjustments were made to the images for these purposes. We measured the area of the TM, thickness of the hair cell epithelium, distance between the TM and hair cell epithelium, thickness of the spiral limbus and OSL, thickness of the RM, and thickness of the bone and SL at the RM. We also measured the penetration depth and image intensity at the soft tissues. The structural measurements were made on slices of the image stacks that were from the middle third of the cochlea. As well, we recorded the pixel intensity values across the internal spiral sulcus.

To measure the area of the TM, the outline of the TM was traced, and the internal area was determined in Photoshop CS4. The thickness of the hair cell epithelium was measured as the distance from the lower edge of the BM to the upper edge of the hair cell epithelium at a point directly lateral to the internal spiral sulcus, perpendicular to the BM. The distance between the TM and hair cell epithelium was defined as the shortest distance between the two that is perpendicular to the BM. The thickness of the spiral limbus and OSL was measured from the RM and spiral limbus connection to the lower edge of the OSL, perpendicular to the OSL. The thickness of the RM was measured at the midpoint, perpendicular to the curvature at that point. Finally, the thickness of the bone and SL was measured at the connection point between the RM and SL, perpendicular to the curvature at that point.

Penetration depth was measured by choosing an A-line that was near the midpoint of where the RM attaches to the spiral limbus. The amount of tissue imaged, as determined by eye, refers to the length of bone and soft tissue minus the length of the fluid-filled space. The image intensity was measured at the apical otic capsule, hair cells, spiral limbus, and perilymph of the ST and was calculated by averaging the pixel intensity within a 10 by 10 pixel box. Weber contrast was calculated by dividing the perilymph intensity (background) from the difference of either the image intensity of apical otic capsule, hair cells, or spiral limbus (signal) and perilymph. This value was then multiplied by 100 and presented as a percentage. Analysis of variance (ANOVA) followed by two-tailed, non-paired Student's t-tests were used to assess for statistically significant differences in measurements of distance, thickness, or image intensity between tissues (P<0.05).

The pixel intensity values across the region of the internal spiral sulcus were recorded from images derived by averaging five consecutive OCT image slices from a single image stack. The intensity values were recorded along a 100 μm line drawn perpendicular to the BM, across the internal spiral sulcus. Two-tailed, non-paired Student's t-tests were used to determine significance between the pixel intensity values from 0 to 10 μm, 45 to 55 μm, and 90 to 100 μm.

### Results

### OCT Image of an Unopened Murine Cochlea

Using our spectral domain OCT system, we first imaged an excised P15 mouse cochlea. A sample X-Z slice and A-line are shown in FIG. 4A and FIG. 4B, respectively. FIG. 4A is an OCT image from a P15 mouse cochlea. The bone and soft tissue structures scatter light and produce a signal that is visible with OCT. In contrast, the surrounding fluid does not produce a visible signal. FIG. 4B is a magnitude and depth plot of the A-line highlighted in line 404 in FIG. 4A. In the OCT image, cochlear structures such as the organ of Corti and stria vascularis can be identified based on our knowledge of cochlear anatomy. These structures can also be identified on the A-line based on their relative depth.

### OCT Image of an Opened Murine Cochlea

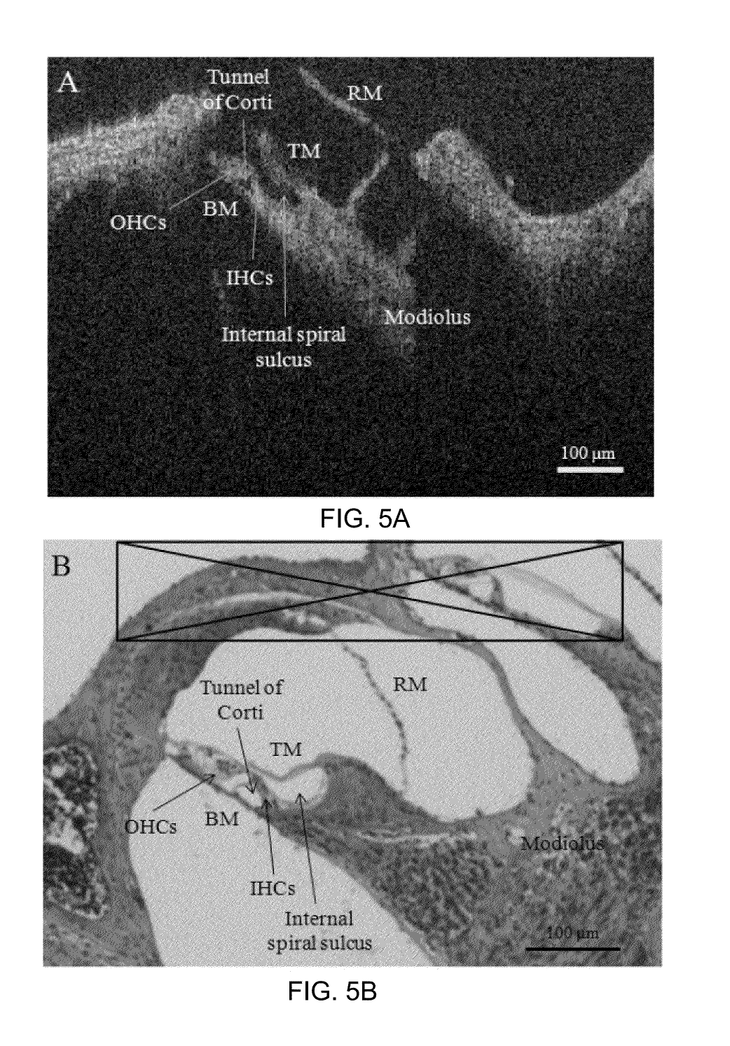

We then imaged normal adult mouse cochleae with the apical otic capsule bone removed to minimize unwanted scattering. A representative OCT image, along with a representative paraffin-embedded histological image of an equivalent region of the cochlea, is shown in FIGS. 5A-B. FIG. 5A is a spectral domain OCT image of an adult mouse organ of Corti (unaveraged) as viewed with the apical otic capsule removed. FIG. 5B is a paraffin-embedded histological section from a P15 mouse cochlea shown for comparison. The box encompasses what is not present in FIG. 5A. In both cases, the basilar membrane (BM), inner hair cells (IHCs), internal spiral sulcus, outer hair cells (OHCs), modiolus, Reissner's membrane (RM), tectorial membrane (TM), and tunnel of Corti are visible. In the OCT image, the RM, BM, TM, and modiolus could be clearly identified. Because the apical otic capsule had been removed, the lateral edge of RM was unattached, opening the scala media. The curvature of the TM was away from the hair cell epithelium. The crevice under the attachment of the TM, the internal spiral sulcus, and the space between the IHCs and OHCs, the tunnel of Corti, were also visible. In the histological image, these structures can also be seen, but are often distorted as a result of the fixation and dehydration process. This was extremely apparent in the case of the TM, which appeared to be thinned dramatically. This is a common problem with cochlear histology and occurs because the TM is composed of ˜97% water. As evident in FIG. 5A, OCT imaging can resolve the soft tissues within the apical turn of the excised, murine cochlea. It also appears to provide a more representative characterization of the in vivo anatomy of the TM than fixed histological sections.

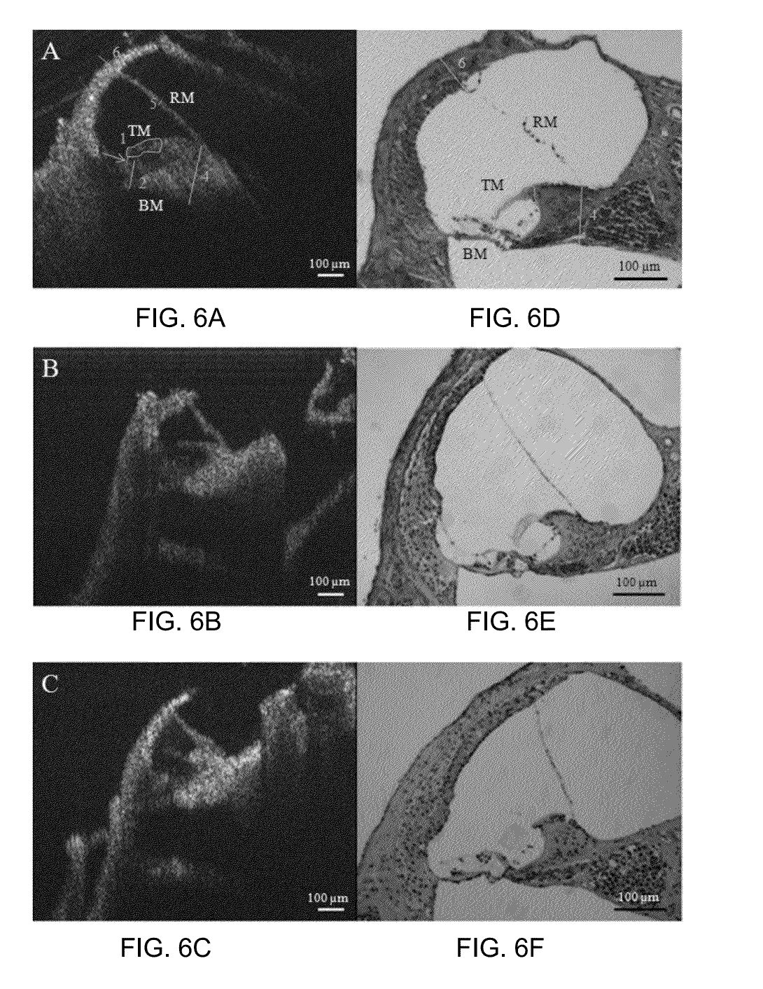

We then imaged cochleae from the three genotypes of transgenic TectaC1509G mice to determine whether OCT can visualize soft tissue changes in the TM anatomy that cause hearing loss. Tecta+/+ mice have a normal TM which attaches to all three rows of OHCs. Tecta+/C1509G mice have a TM which attaches to only the first row of OHCs and suffer moderate hearing loss. TectaC1509G/C1509G mice have a TM that does not attach to any OHCs and suffer profound hearing loss. To ensure more natural cochlear anatomy, we kept the spiral ligament and Reissner's membrane connection intact during the dissection, ensuring that the scala media was not opened. FIGS. 6A-F shows representative OCT images of the cochlea. FIGS. 6A-C show OCT histological sections and FIGS. 6D-F show paraffin-embedded of cochleae from FIGS. 6A,D Tecta+/+, FIGS. 6B,E Tecta+/C1509G, and FIGS. 6C,F TectaC1509G/C1509G. The OCT image of the Tecta+/+ gives the locations of where we made the measurements for (1) the area of the tectorial membrane (TM), (2) thickness of the hair cell epithelium, (3) the distance between the TM and hair cell epithelium, (4) the thickness of the spiral limbus and OSL, (5) the thickness of the RM, and (6) the thickness of the bone and SL at its junction with Reissner's membrane (RM). Since the bone was opened in the OCT images, the actual measurement of the bone and SL thickness was made at another slice of the image stack. Depicted is the approximation of that thickness in the current slice. We found that the distance between the TM and hair cell epithelium increased with the severity of the mutation, from an average of 16.31±0.63 μm in Tecta+/+ to 23.45±0.76 μm in Tecta+/C1509G and 54.53±4.46 μm in TectaC1509G/C1509G (P<0.05; Table 1).

| | | | Tecta^{+/+} | | Tecta^{+/C1509G} | | Tecta^{C1509G/C1509G} | A | B | C |
|---|---|---|---|---|---|---|---|---|---|---|
| TM area (μm²): | OCT | * | 3862.5 ± 144.7 | | 4272.92 ± 72.9 | * | 5443.75 ± 139.9 | ¥ | ¥ | ¥ |
| | Histology | | 1094.31 ± 322 | | 3461.183 ± 399 | | 3461.29 ± 340.9 | ¥ | ¥ | |
| Hair cell epithelium | OCT | * | 115.14 ± 3.73 | * | 142.94 ± 0.99 | * | 139.16 ± 3.44 | ¥ | ¥ | |
| thickness (μm): | Histology | | 44.37 ± 2.31 | | 53.30 ± 1.69 | | 57.37 ± 4.39 | ¥ | ¥ | |
| Distance between TM and | OCT | | 16.31 ± 0.63 | * | 23.45 ± 0.76 | * | 54.53 ± 4.46 | ¥ | ¥ | ¥ |
| hair cell epithelium (μm): | Histology | | 17.28 ± 4.93 | | 10.05 ± 2.33 | | 24.24 ± 7.94 | | | |
| Spiral limbus and OSL | OCT | * | 204.57 ± 6.36 | * | 218.62 ± 5.87 | * | 224.36 ± 9.77 | | | |
| thickness (μm): | Histology | | 151.22 ± 9.69 | | 174.13 ± 9.08 | | 161.26 ± 11.86 | | | |
| RM thickness (μm): | OCT | * | 20.03 ± 0.88 | * | 22.66 ± 1.84 | * | 22.28 ± 0.33 | | | |
| | Histology | | 6.21 ± 1.02 | | 6.32 ± 0.47 | | 7.42 ± 0.88 | | | |
| Bone and SL | OCT | | 105.33 ± 4.65 | | 109.4 ± 6.24 | | 106.24 ± 6.32 | | | |
| thickness (μm): | Histology | | 112.88 ± 14.33 | | 102.9 ± 7.66 | | 109.67 ± 9.77 | | | |

Table 1 shows the measurements of the soft tissue structures within the cochlea. All values are mean±SEM. Measurements from OCT and histological images are as labeled. * denotes statistical significance between the measurement from OCT and histology. ¥ denotes statistical significance among genotypes, where A is between Tecta^{+/+} and Tecta^{+/C1509G}, B is between Tecta^{+/+} and Tecta^{C1509G/C1509G}, and C is between Tecta^{+/C1509G} and Tecta^{C1509G/C1509G}.

To determine if differences exist between OCT and histological images, we made measurements (mean±SEM) of (1) the area of the TM, (2) the thickness of the hair cell epithelium, (3) the distance between the TM and hair cell epithelium, (4) the thickness of the spiral limbus and OSL, (5) the thickness of the RM, and (6) the thickness of the bone and SL at its junction with RM. Table 1 summarizes the data. Measurements were made on six different images from two different cochleae, except in the OCT case of Tecta+/+ which were made from three different cochleae.

When comparing the measurements made on the OCT images between genotypes, there were also differences between the TM area and hair cell epithelium thickness. For the TM area, the measurement increased with the severity of the mutation. For the hair cell epithelium thickness, the Tecta+/+ was less than the Tecta+/C1509G and TectaC1509G/C1509G; however, the Tecta+/C1509G and TectaC1509G/C1509G were not different from each other. The differences in the TM area and hair cell epithelium thickness were reflected in the measurements from the histological images as well, except for between Tecta+/C1509G and TectaC1509G/C1509G. Importantly, there were no differences in the spiral limbus and OSL thickness, RM thickness, and bone and SL thickness when comparing between genotypes in both OCT imaging and histology. These were not expected to change. Thus, we conclude that OCT imaging can distinguish between TM differences in mice that contain a mutation responsible for hearing loss in humans.

### Unopened Cochlea During Development and in Adulthood

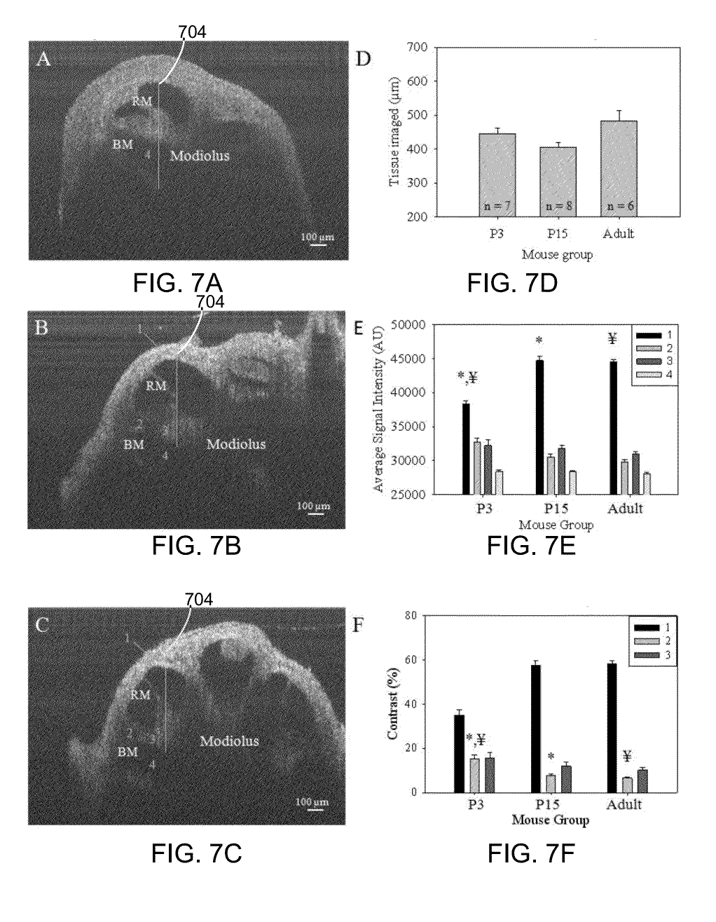

The mouse cochlea is only partially formed at birth. At P3, the TM is still attached to the hair cell epithelium along its entire width, because the internal spiral sulcus and tunnel of Corti have not formed yet. The otic capsule surrounding the cochlea has not yet undergone endochondral ossification and remains cartilaginous. As such, it should scatter light less than in adult mice. By P15, the organ of Corti is fully mature, and the otic capsule has partially ossified. The adult mouse cochlea (>P30) has a more ossified otic capsule. Therefore, we studied cochleae from P3, P15, and adult mice to assess the abilities of our system to visualize developmental changes in soft tissue morphology and to understand the impact of otic capsule ossification on image quality. Unaltered, representative images of the cochlea are shown in FIGS. 7A-C of a P3 (FIG. 7A), P15 (FIG. 7B), and adult (FIG. 7C) cochlea. In all cases, the Reissner's membrane (RM), basilar membrane (BM), and modiolus are visible. An example of the A-line 704 chosen for measuring the amount of tissue imaged is shown. Examples of the 10 by 10 pixel boxes used to calculate the signal intensity in a given region are also shown, by the reference numbers 1-4. FIG. 7D is a graph of the amount of tissue imaged. FIG. 7E is a graph of the average signal intensity in different regions. FIG. 7F is a graph of the contrast percentage of regions 1, 2, and 3. The number of samples is noted in FIG. 7D, and statistical significance is noted in each of the graphs by a paired * or ¥. In all of the image stacks, the BM and the modiolus were visible in at least 80% of the apical cochlear turn. Looking at an A-line 704 that was near the attachment of RM to the spiral limbus, the total depths of tissue imaged in the P3, P15, and adult cochleae were 445.71±16.53 μm (n=7), 405.63±14.28 μm (n=8), and 482.5±30.02 μm (n=6, mean±SEM), respectively. There was no statistical significance between these measurements, as summarized in FIG. 7D.

The image quality of the soft tissues was reduced in the P15 and adult mouse. This is illustrated in FIG. 7E. The graph depicts the average signal intensity of 10 by 10 pixel boxes. The location where the signal intensity was measured is shown roughly by the numbers 1, 2, 3, and 4 in (FIGS. 7A-C). The regions encompass a portion of the apical otic capsule, the hair cells and supporting cell region, the spiral limbus, and the ST, respectively. As would be expected, there was a statistically significant increase in the overall signal in the apical otic capsule with age, and no difference in the signal from the ST. Importantly, there was a decrease in the signal from the hair cells at P15 and adult; this is more clearly seen when looking at the contrast in FIG. 7F. There were, however, no statistically significant differences in the signal intensity from the spiral limbus between the different age groups. In the cochlea of the P3 mouse, since the bone has not fully calcified, the inner cochlear structures are better defined.

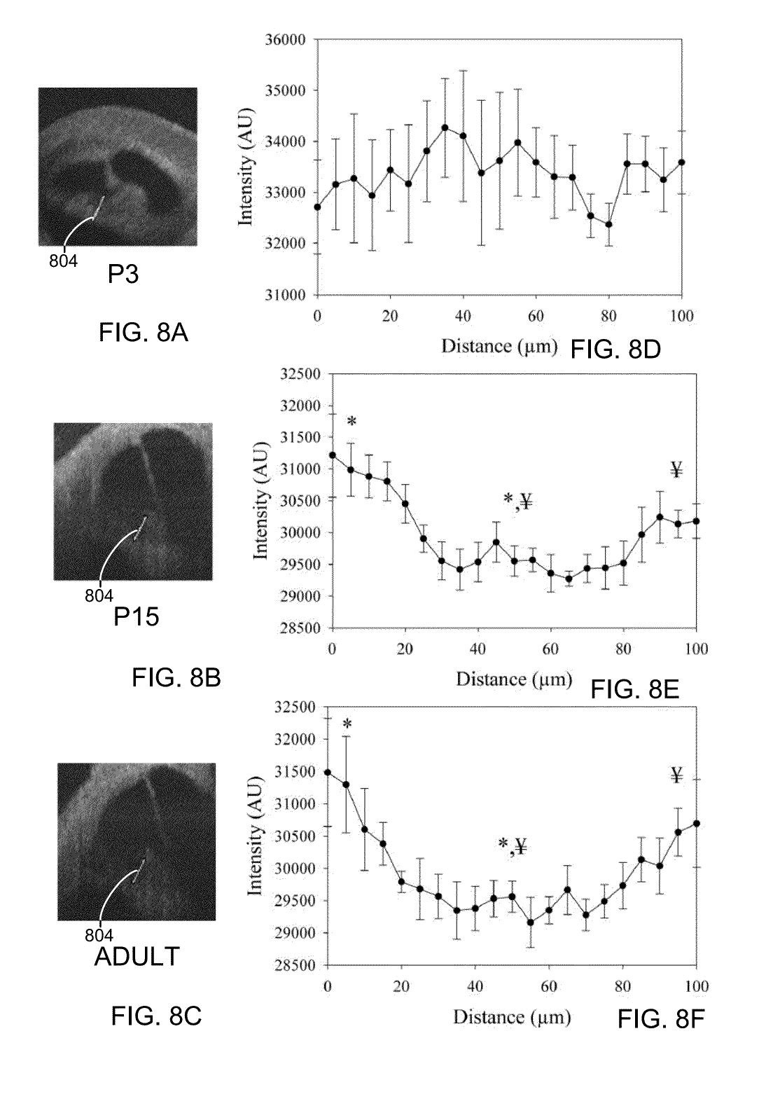

Additionally, the TM does not lift from the BM in the mouse until after P3; this is shown in the P3 OCT image by a lack of the internal spiral sulcus. The pixel intensity values across the region of the internal spiral sulcus are graphed in FIGS. 8A-F. FIGS. 8A-F show the average intensity OCT images from five slices from the cochlea of the P3 (FIGS. 8A, D) (n=7), P15 (FIGS. 8B,E) (n=8), and adult mouse (FIGS. 8C,F) (n=6). The pixel intensity across the internal spiral sulcus, depicted by line 804 (100 μm), is graphed. The zero position is closer to the basilar membrane (BM). Statistical significance is noted in each of the graphs by a paired * or ¥. There was a decrease in the signal around the 50 μm position, representing the fluid filled internal spiral sulcus, in the P15 and adult. The higher signal at the 0 μm position is the organ of Corti and osseous spiral lamina; the higher signal at the 100 μm position is the spiral limbus and TM. Therefore, OCT provides the ability to observe a critical, yet subtle change in the normal development of cochlear anatomy.

In using an embodiment of the invention, it was found that spectral domain OCT used in an embodiment of the invention can provide high-resolution images of the soft-tissue structures critical to normal hearing. Using freshly-excised mouse cochleae, an embodiment of the invention provides routine visualization and assessment of several critical structures, including Reissner's membrane, the basilar membrane, the hair cell region, the tectorial membrane, the spiral ligament, the spiral limbus, and the modiolus. Of greater interest is the ability of this embodiment of the invention to identify anatomic malformations that define the pathophysiology of hearing loss in a mouse model of human disease. In these experiments, this embodiment of the invention was used to image the cochlea at discrete time points throughout the development of the mouse cochlea. Monitoring the morphology of the cochlear soft tissue during the developmental timeline is important not only for our understanding of inner ear maturation, but also for understanding how problems in maturation can lead to congenital malformations. One concern about using this embodiment of the invention to study the inner ear is the impact of the surrounding bone, which is highly scattering. However, our study has shown that while otic capsule ossification affects image quality to a degree, it does not substantially impact the ability to study the internal soft tissue structures in adult mice.

Furthermore, this embodiment of the invention overcomes many of the problems associated with the substantial histological artifact that occurs with fixation, decalcification, dehydration, and embedding of the cochlea. In general, the measurements made from tissue processed by fixed histology were less than from fresh tissue imaged by this embodiment of the invention. We attribute the majority of the differences between the measurements of the soft tissues in the OCT images and in the histological images to dehydration-induced shrinkage. Indeed, a previous study in gerbils has shown that the TM cross-sectional area, as well as that of other cochlear tissues, can shrink dramatically depending on the dehydration protocol. We should note, however, that in the opened cochlea, the TM is no longer in its native environment and its shape can change depending on the ionic imaging solution that is used. Consistent with this notion, structures that have lower water content had similar measurements between the two imaging modalities. In particular, the thickness of the bone and SL measurements in all three genotypes were not different. Imaging time is another benefit from this embodiment of the invention compared to traditional histological sectioning. An entire cochlea can be imaged using this embodiment of the invention within a couple of minutes, whereas the fixation, decalcification, paraffin embedding, sectioning, and imaging associated with histology would typically take a week or more to accomplish.

Most importantly, analysis of the images produced by this embodiment of the invention provided important findings that could not be made by an analysis of only the histological images. When comparing the genotypes, measurements from the images produced by this embodiment of the invention showed statistically significant differences in the distance between the TM and hair cell epithelium. This was not evident from the analysis of the histological images. Furthermore, in our original description of the Tecta mutant mouse, we decided against measuring the thickness of the hair cell epithelium in the histological images because we thought those measurements would be tainted by artifact. Our current measurements from both this embodiment of the invention and histological images suggest that there is indeed an increase in the thickness of the hair cell epithelium in the Tecta+/C1509G and TectaC1509G/C1509G. This may reflect the fact that Tecta+/C1509G and TectaC1509G/C1509G mice have an upregulation of the prestin protein within their OHCs. Prestin is a motor protein that produces force to amplify the sound pressure waves within the cochlea, and indeed in these mutants, increased prestin results in increased vibratory amplitudes of the organ of Corti. Alternatively, the structure of the hair cell epithelium may have developed differently because of the altered biophysical properties of the overlying malformed TM

The images from this embodiment of the invention of the mouse cochlea at different ages, which were taken without removing or thinning the cochlear otic capsule bone, revealed the expected compositional and structural changes associated with development. These include the endochondral ossification of the otic capsule and the resorption of the inner sulcus cells, freeing up the middle region of the TM. The latter is a key developmental milestone in achieving a functional cochlea.

System in an Embodiment of the Invention with a Sound Generator

The ability to measure how sound causes structures within the ear to vibrate is severely limited. Hearing loss often has its origin in pathological processes that alter these normal vibratory patterns. The resolution, speed, sensitivity, and ability to image through turbid media provided by an embodiment of the invention allows high-fidelity in vivo images of ear morphology and function to be provided by this embodiment. Such images are useful in building our understanding of hearing loss in animal models, aid diagnosis in humans, and potentially guide surgical intervention.

The exquisite phase sensitivity of an embodiment of the invention can be exploited to measure the extremely small periodic mechanical motions of the ear. Measured picometer scale sensitivities compare favorable to the nanometer scale motion expected in the middle and inner ear. The embodiment of the invention with a phase sensitive OCT system can be utilized as a high-resolution non/minimally invasive vibrometer.

The similarity of mouse ear function to humans as well as the ability to generate mice that contain hearing loss mutation has made mice one of the most prevalent animal models of hearing. Human hearing ranges between 20 Hz-20 kHz, however mice hearing ranges between 4-90 kHz. The high-frequency range poses a technical problem for imaging systems.

Based on the Nyquist sampling theorem, in order to measure a 90 kHz signal, the sampling rate must be at least 180 kHz. The sampling rate for measuring motion in an embodiment of the invention with a spectrometer based OCT system is the line-scan camera's line-rate. For most systems, the line-rate is below 60 kHz and largely limited by the readout time. A Nyquist frequency of 30 kHz, while adequate for humans, leaves two thirds of the hearing spectrum of mice unreachable. Shorter read-out times are available with CMOS cameras however they also have reduced bit depth and increased noise, leading to lower phase sensitivity.

An embodiment of the invention enables the use of slow line-rate, yet high-sensitivity, CCD line-scan cameras, which still interrogates the entire range of the mouse hearing spectrum. This embodiment exploits the periodic nature of the mechanical motion of the ear by phase-locking the camera triggering to the acoustic stimulation of the ear. It is analogous to the coherent interleaved sampling technique used in oscilloscopes which phase-locks the sample clock to the bit clock.

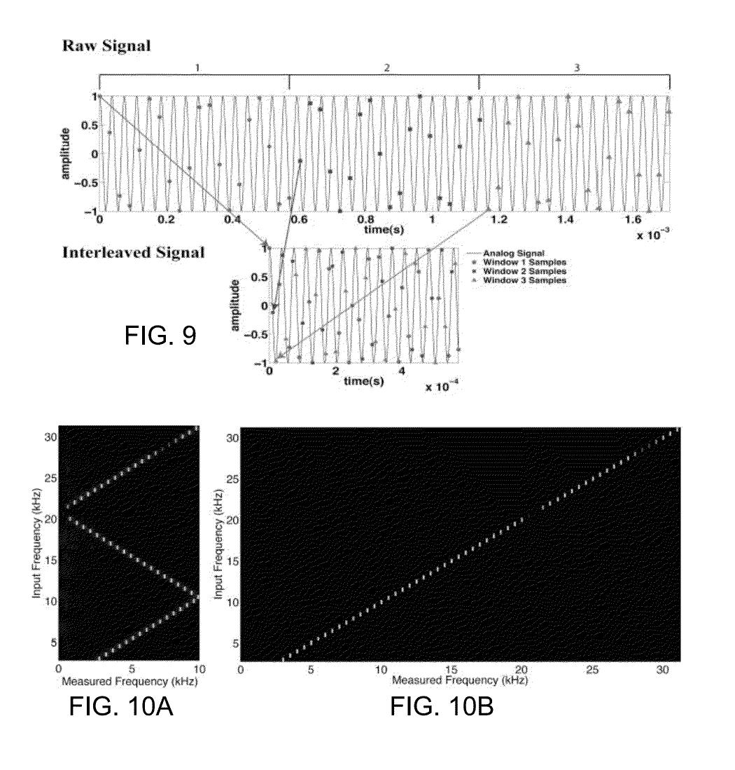

Consider the following example shown schematically in FIG. 9. The analog signal (interferometric phase) is divided into 3 time intervals or windows. Within each window, the line-scan camera is triggered at rate fₛ. Each trigger initiates an integration time and corresponds to one sample. The triggers in each window are phase shifted from the previous window by τₛ/3+φ relative to the previous window, where φ is given by φ=ceil(τs/τ)τ, τₛ is the sampling period, and τ is the period of the signal. The additional phase factor, φ, ensures that we do not attempt to sample faster than fₛ and that the sampling remains in phase with the analog signal. When the samples from the three windows are interleaved the effective sampling is 3fₛ, which leads to a Nyquist frequency of 3/2fₛ.

More generally, assume we have a periodic signal S(t) that exists over a time interval T. If we divide T into N windows, the phase shift between adjacent windows is given by τₛ/N+φ. The coherently interleaved signal is then

S(t)=Σᵢ₌₁ⁿΣⱼ₌₁^{N}a_{i,j}t_{i,j} (1)

where n is the number of samples per window and a_{i,j} is the amplitude of the signal at time t_{i,j}. The original signal is simply equation 1 with the order of summations swapped. The coherently interleaved signal will then have sampling and Nyquist frequencies of Nfₛ and Nfₛ/2, respectively. The number of windows may be arbitrarily increased in order to increase the Nyquist frequency.

Practically, the signal will be degraded due to rolloff associated with the integration time as the signal frequency, f, approaches (tᵢₙₜ)⁻¹. If we assume the integration time of the camera may be approximated by a rectangular function with width tᵢₙₜ, then the rolloff is given by sinc(tᵢₙₜπf). Under these conditions, the signal strength will be exactly 0 at f=(tᵢₙₜ)⁻¹. Furthermore, the signal will be reduced at f=½(tᵢₙₜ)⁻¹ and f=¾(tᵢₙₜ)⁻¹ by ˜40% and ˜60%, respectively, hence the rolloff significantly reduces the useful frequency range.

In order to use the coherently interleaved sampling algorithm, the period of the signal must be known a priori. Fortunately, this is the case for hearing tests on humans or animals. The stimulus is a pure sine wave tone played from a calibrated, low-distortion speaker. While there are distortion products generated within the ear, the resulting frequency spectra are fairly simple. Aliased frequencies can be readily identified by recording two sets of data with slightly different sampling frequencies and looking for peaks which change frequency. The true frequency of the aliased peaks may be estimated from this data as well and the estimate of signal period revised accordingly. The period of the total signal is simply the least common multiple of the periods of each component signal.

This embodiment of the invention uses a spectrometer based OCT system to test and verify the algorithm described above. The system used a 40 nm bandwidth super luminescent diode centered at 830 nm as a source. The custom built spectrometer had a maximum line rate of 28 kHz at an integration time of 5 μs. A 2×2 (50:50) fused fiber coupler formed the backbone of a Michelson type interferometer. The sample beam was scanned across the sample by using a 2-D galvonometer based mirror scanner. The lateral and axial resolutions were 14 μm and 8 μm (in air), respectively.

As a proof of concept demonstration of the algorithm, this embodiment of the invention was used to image a piezo-electric element. The piezo was driven with a sinusoidal voltage which induced a small sinusoidal vibration. In these experiments, the amplitude of the motion was maintained at ˜0.23 radians by adjusting the amplitude of the driving voltage at each frequency. At 28 and 28.5 kHz, the power of the waveform generator was insufficient to maintain the amplitude.

For each drive (input) frequency an M-scan was acquired of the piezo using 3 windows with 400 lines per window. The integration time was set at 12 μs which resulted in a sampling rate of 20.83 kHz and a Nyquist frequency of 10.42 kHz. Interleaving the three windows yielded a Nyquist frequency of 31.25 kHz.

Each raw data set was processed using the following algorithm in Matlab. The DC component of the spectral interferogram was removed by subtracting off the DC signal synthesized by averaging all of the spectra in the M-scan and then low-pass filtering with a digital filter. The remaining interferometric part of the signal was resampled in k-space by using a cubic spline interpolation. A fast-Fourier transform yielded the M-scan.

In order to extract the periodic motion of the piezo, the phase of the M-scan at the depth corresponding to the peak intensity was further processed. The phase in each of the 3 windows was independently unwrapped and high-pass filtered with a cutoff frequency of 300 Hz. The high-pass filter served to remove any DC term in the phase and low frequency phase drift. The unwrapped filtered phase was then interleaved as outlined above and multiplied by a Hanning window before calculating the frequency spectrum via fast-Fourier transform. The results for input frequencies of 3-31 kHz with 0.5 kHz steps are shown in FIGS. 10A-B, where the ordinate is the input frequency, the abscissa is the measured frequency, and the graylevel corresponds to the amplitude of the frequency spectrum. FIGS. 10A-B are the results without and with coherent interleaving, respectively. The gap centered on the sampling frequency is due to the highpass filtering of the unwrapped phase. Signals at the sampling frequency are aliased back to DC and therefore filtered out in this step. As expected, in FIG. 10A, when the input frequency exceeds the Nyquist frequency we observe aliasing of the signal. In contrast, if we interleave the samples from the three windows we are able to reliably measure the frequency content of the signal up to the new Nyquist frequency, 31.25 kHz.

The relative phase of the vibrational motion is also an important biometric that can be measured in the mouse ear using this embodiment of the invention. Theoretically the algorithm should not alter the measured vibrational phase. We tested this contention using the piezo. Indeed a phase shift in the drive frequency of the piezo was correctly reported in the phase of the Fourier transform. We conclude that the relative phase is preserved by the algorithm used in this embodiment of the invention and correctly reported in the Fourier transform.

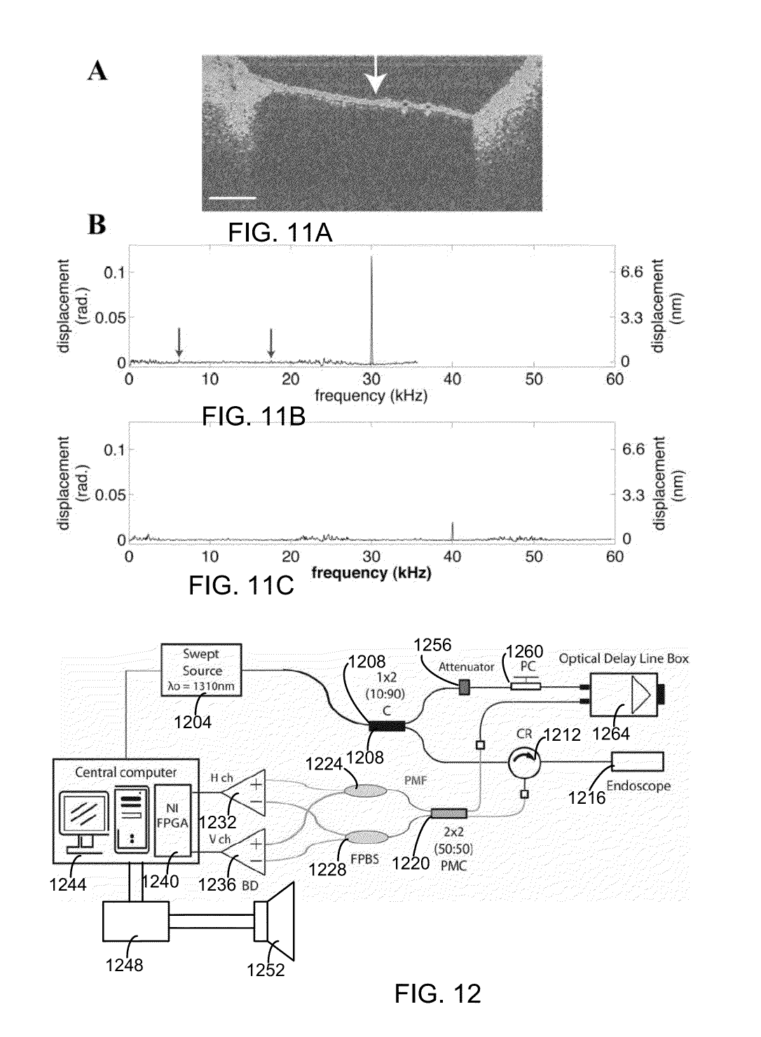

We have further evaluated the developed algorithm in this embodiment of the invention by imaging vibrations in the tympanic membrane of a euthanized adult mouse. The outer ear of the mouse was removed in order to provide unfettered access to the middle ear with our current imaging optics. To stimulate vibration of the tympanic membrane a tone was played through a speaker (Pyle electronics, PSN1165) placed within 12 inches of the mouse. A cross-sectional image (B_{scan}) through the tympanic membrane is shown in FIG. 11A. The arrow indicates the approximate area where the vibrational response was measured.

The data was collected as before except the camera integration time was 8 μs which resulted in a sampling rate of 23.8 kHz and Nyquist frequency of 11.9 kHz. Two examples of measured vibrational frequency spectra are shown in FIGS. 11B-C. The example in FIG. 11B was stimulated at 30 kHz and recorded with 3 windows. The example in FIG. 11C was stimulated at 40 kHz and recorded with 5 windows. The 30 kHz cutoff frequency of the speaker in this embodiment of the invention prevented higher frequency measurements. In both cases, the frequency content matches what we would expect from stimulation of the tympanic membrane with a pure tone.

Two other features worthy of note are provided by this embodiment of the invention in addition to the strong signals at the stimulation frequency. First, the relative phase noise is increased around the sampling frequency and its harmonics. This is due to the fact that the noise is not coherently interleaved and is therefore effectively aliased. For instance some of the noise around DC is shifted to either side of the sampling frequency and its harmonics. The noise power that is shifted depends on the relationship of the particular noise frequency to the interleaved sampling parameters; hence the noise power is not perfectly mirrored at the sampling frequency and its harmonics. The second feature is visible in FIG. 11B. Arrows at 6.2 kHz and 17.6 kHz indicate two small peaks due to aliasing of the 30 kHz signal. In this example the aliased peaks are approximately 125 times weaker than the main peak at 30 kHz. In general, both types of artifacts were visible in the data taken with the piezo element as well.

The added noise around the sampling frequency and harmonics is a fundamental consequence of the algorithm. Suppressing the phase noise by implementing either a common mode interferometer or a phase reference in an embodiment of the invention would help mitigate the effects. A second pragmatic approach in another embodiment of the invention would be to simply vary the sampling frequency such that the signal at the stimulation frequency is in a low noise region. The standard deviation of the noise in a quiet region of the spectrum (13-18 kHz) was 2.8×10−4 radians (19 pm). One or both approaches will be sufficient to reduce phase noise to acceptable levels for our purposes.

In principle, the algorithm in an embodiment of the invention should completely suppress the aliased peaks. In practice, they would become visible seemingly at random when recording multiple data sets in succession with the same parameters. Given the short acquisition time, recording several data sets in order to get one without the aliased peaks was not particularly burdensome. Nevertheless, in order to investigate the source of the aliased peaks and to test how robust the algorithm was to various sources of noise, we built a model of the signal and tested the algorithm using Matlab. The model was equivalent to interference from a reflector oscillating at a single frequency, illuminated with a Gaussian source, and recorded with zero integration time.

In modeling an embodiment of the invention, we systematically added random noise to the sine wave amplitude and phase, and trigger times. We also modeled phase drift by adding a time dependent offset to the phase using a linear and quadratic term. No aliased peaks were apparent. We were only able to reproduce the observed aliased peaks when we introduced a systematic error into the trigger times for one window. For a system with fₛ=33.3 kHz and three windows a 1% (100 ns) error in the phase shift in one window produced aliased peaks that were 0.5% of the amplitude of the signal peak. Based on this result, we speculate that the observed aliased peaks are due to transient systematic errors in the response of the camera to the trigger signal.

Therefore, an embodiment of the invention provides a robust algorithm that enables the measurement of the vibratory response of the mouse ear over its entire spectrum (4 kHz-90 kHz). The algorithm uses a coherent interleaving technique that phase-locks the acoustic stimulation to the line-camera trigger. We have demonstrated the technique by measuring the vibratory response of the mouse tympanic membrane upon stimulation with a pure tone. Modeling of the algorithm indicates that it is robust to noise in the amplitude, phase, and trigger timing, however it is susceptible to systematic error in the trigger timing.

### System With an Endoscope

FIG. 12 is a schematic illustration of another embodiment of the invention that uses a spectral domain OCT system with an endoscope and a sound system. The light source 1204 consisted of swept laser with 50 kHz sweep rate centered at 1310 nm. The light directed into a 1×2 (10:90) fiber coupler 1208. One of the output ports was coupled to a circulator 1212, which directs some of the light to an endoscope 1216, which directs light to scan an inner ear. The circulator 1212 directs light reflected from the inner ear and received by the endoscope 1216 to a 2×2 (50:50) fiber-fused coupler and polarization maintaining coupler 1220. The fiber-fused coupler and polarization maintaining coupler 1220 provides output to a first fiber-optic beam splitter 1224 and a second fiber-optic beam splitter 1228. The output of the first fiber-optic beam splitter 1224 is provided to a positive input of a horizontal channel differential amplifier 1232 and a positive input of a vertical channel differential amplifier 1236. The output of the second fiber-optic beam splitter 1228 is provided to a negative input of a horizontal channel differential amplifier 1232 and a negative input of a vertical channel differential amplifier 1236. The outputs of the horizontal and vertical channel differential amplifiers 1232, 1236 are provided as input to a Field Programmable Gate Array (National Instruments) 1240, which is part of a central computer 1244. A sound system 1248 is connected to the central computer 1244. At least one speaker 1252 is connected to the sound system 1248. The speaker may be a regular speaker or an ear phone or a special speaker, such as a speaker tube, connected to the endoscope. The sound system 1248 and speaker 1252 may be separate from the central computer 1244 or may be integrated with the central computer 1244. Some of the output from the 1×2 fiber coupler 1208 is passed through an attenuator 1256 and through an in-line polarization controller 1260 to an optical delay line box 1264. The optical delay line box 1264 allows an adjustment to equalize the beam paths through which the light travels. The output of the optical delay line box 1264 is provided as input to the 2×2 (50:50) fiber-fused coupler and polarization maintaining coupler 1220.

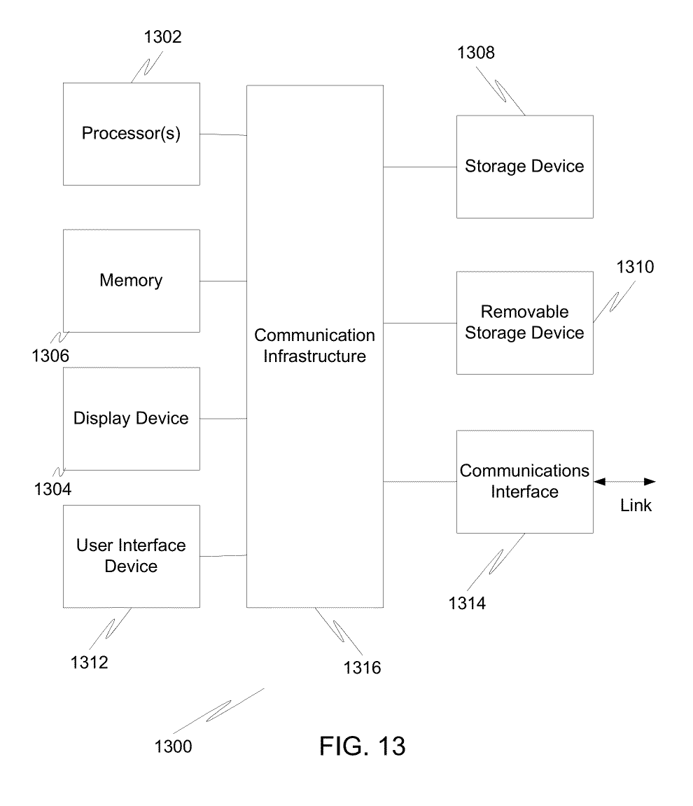

FIG. 13 is a high level block diagram showing a computer system 1300, which is suitable for implementing a central computer 1244 used in embodiments of the present invention. The computer system may have many physical forms ranging from an integrated circuit, a printed circuit board, and a small handheld device up to a huge super computer. The computer system 1300 includes one or more processors 1302, and further can include an electronic display device 1304 (for displaying graphics, text, and other data), a main memory 1306 (e.g., random access memory (RAM)), storage device 1308 (e.g., hard disk drive), removable storage device 1310 (e.g., optical disk drive), user interface devices 1312 (e.g., keyboards, touch screens, keypads, mice or other pointing devices, etc.), and a communication interface 1314 (e.g., wireless network interface). The communication interface 1314 allows software and data to be transferred between the computer system 1300 and external devices via a link. The system may also include a communications infrastructure 1316 (e.g., a communications bus, cross-over bar, or network) to which the aforementioned devices/modules are connected.

Information transferred via communications interface 1314 may be in the form of signals such as electronic, electromagnetic, optical, or other signals capable of being received by communications interface 1314, via a communication link that carries signals and may be implemented using wire or cable, fiber optics, a phone line, a cellular phone link, a radio frequency link, and/or other communication channels. With such a communications interface, it is contemplated that the one or more processors 1302 might receive information from a network, or might output information to the network in the course of performing the above-described method steps. Furthermore, method embodiments of the present invention may execute solely upon the processors or may execute over a network such as the Internet in conjunction with remote processors that shares a portion of the processing.

The term “non-transient computer readable medium” is used generally to refer to media such as main memory, secondary memory, removable storage, and storage devices, such as hard disks, flash memory, disk drive memory, CD-ROM and other forms of persistent memory and shall not be construed to cover transitory subject matter, such as carrier waves or signals. Examples of computer code include machine code, such as produced by a compiler, and files containing higher level code that are executed by a computer using an interpreter. Computer readable media may also be computer code transmitted by a computer data signal embodied in a carrier wave and representing a sequence of instructions that are executable by a processor.

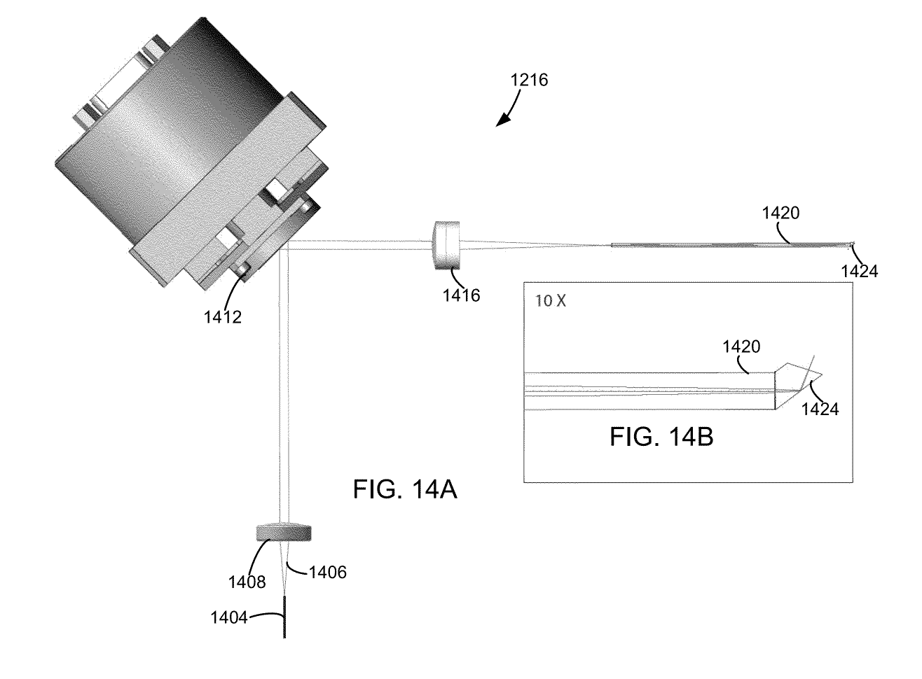

FIG. 14A is a more detailed schematic view of an endoscope 1216. The endoscope forms the sample arm of the OCT system. The endoscope comprises a single mode fiber 1404, a collimating lens 1408, a steering minor 1412, a focusing lens 1416, a Gradient Index (GRIN) lens 1420, and an end piece 1424 at the end of the GRIN lens 1420. FIG. 14B is a more detailed illustration of the GRIN lens 1420 and end piece 1424. In this embodiment of the invention, the end piece 1424 is attached directly to an end of the GRIN lens 1420. Preferably, the end piece 1224 is attached by an adhesive, such as epoxy. In other embodiments the GRIN lens 1420 and end piece 1224 may form a single object. In other embodiments, the various components of the endo scope may be separated by optical fiber or another optical transmission material. The single mode fiber 1404 is an optical fiber that connects the endoscope to the main part of the OCT system and passes light beams 1406 between the OCT system and the endoscope. The collimating lens 1408 collimates the output of the single mode fiber 1404. In this embodiment this is done by placing the face of the fiber in the back focal plane of the collimating lens 1408. In this embodiment, the steering mirror 1412 is a fast scanning minor that is a voice coil mirror that can deflect in both x and y. The steering mirror 1412 lies in the back focal plane of the focusing lens 1416. The focusing lens 1416 focuses the collimated light to a point outside the GRIN lens 1420. The GRIN lens 1420 images the focal spot made by the focusing lens onto the object being imaged. In this embodiment, the magnification of the GRIN lens 1420 is 1. In other embodiments the magnification of the GRIN lens is greater than 1 to provide an increased field of view. In this embodiment, the end piece 1424 is a turning prism that uses a silver coated side to direct light at the appropriate angle for viewing the inner ear through a round or oval window. In another embodiment, the end piece has a transmission surface that directs light to the inner ear and then receives light from the inner ear and directs it through the endoscope. Preferably, the GRIN lens 1420 and the end piece 1424 have a diameter less than 2 mm to allow the GRIM lens 1420 and end piece 1424 to be inserted in an incision in tympanic membrane or a temporal bone to allow the end piece 1424 to approach the inner ear.

### Method

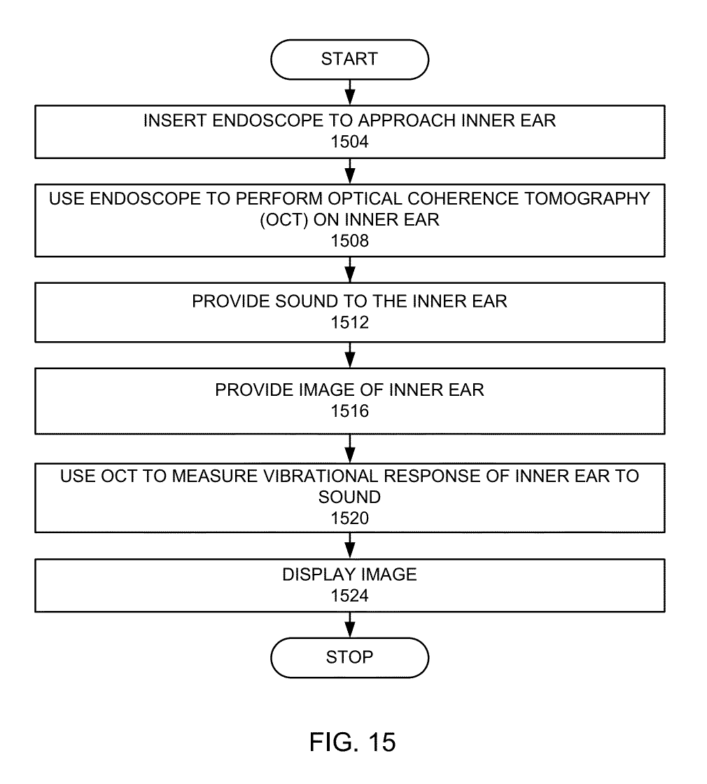

FIG. 15 is a high level flow chart for a method of examining an inner ear using an embodiment of the invention which comprises an endoscope. An endoscope is inserted to approach the inner ear (step 1504). In the preferred embodiments, an end of the endoscope is inserted through the tympanic membrane or temporal bone or into the ear canal adjacent to the tympanic membrane. Preferably, the end of the endoscope is within a distance of 1 cm from the inner ear. More preferably, there is nothing between the end of the endoscope and the inner ear that would prevent performing spectral domain optical coherence tomography directly on the inner ear.

The endoscope is used to perform OCT on the inner ear (step 1508). This may be accomplished by providing a light beam through the endoscope to the inner ear, receiving light reflected from the inner ear, and using the received light to create an image of the inner ear.

Sound is provided to the inner ear (step 1512). In this embodiment, the central computer 1244 sends a command to the sound system 1248 to generate a tone through the speaker 1252. The speaker generates a tone of one or more frequencies. Preferably, the central computer knows at least one dominant frequency in the tone.

An image of the inner ear is provided using the OCT (step 1516). At least one dominant frequency in the tone is used to measure vibrational response of the inner ear to sound (step 1520). In an embodiment of the invention, a first inverse Fast Fourier Transform (FFT) is applied to the spectral interferogram to convert from spatial frequency to space and then a second FFT is applied to the data to provide the data in the frequency domain. The data is analyzed in view of the dominant frequency. The image of the inner ear is displayed (step 1524).

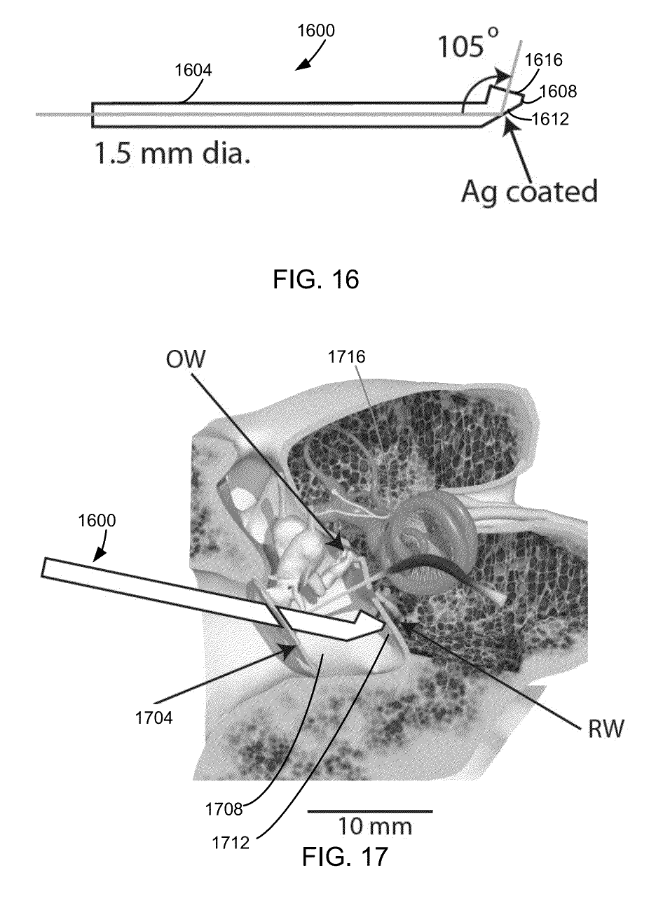

FIG. 16 is a schematic view of another endoscope 1600 used in another embodiment of the invention. In this embodiment the endoscope 1600 comprises a wave guide 1604 and an end piece 1608, where the wave guide 1604 and end piece 1608 are formed from a single piece of material to remove any interface between the wave guide and end piece. The end piece 1608 has an angled and silver coated side 1612 to bend light travelling down the wave guide 1604 at a 105° angle. An end window 1616 has a surface that is close enough to perpendicular to light reflected from the angled and silver coated side 1612 to avoid total internal reflection. In this embodiment, the wave guide 1604 is a GRIN lens and has a diameter of 1.5 mm.

FIG. 17 is a schematic view of the endoscope 1600 in use in an ear. The endoscope is place through an incision in the tympanic membrane 1704 into the tympanic chamber 1708. The end piece 1608 remains in the tympanic chamber 1708. Light is transmitted through the end window 1616 and through the round window membrane or oval window footplate 1712 to the inner ear 1716, where the light is focused to scan and generate an image of the inner ear 1716. In this embodiment, the image plane where the light is focused is 4 mm from the end window 1616. In other configurations where the endoscope passes through the tympanic membrane, the focal plane is between 0 mm and 8 mm from the end window. In other embodiments where the endoscope passes through the tympanic membrane, the end piece bends the light at an angle between 0° to 130°. More preferably, the end piece bends the light at an angle of between 90° to 120°. The exact angle to be selected will depend upon the approach angle through the ear canal, the position of the incision within the tympanic membrane, and the region of the inner ear to be imaged. All of these factors can vary because of patient-specific factors and their disease. For example, scar tissue within the ear drum will affect the position of the incision to be place within the eardrum.

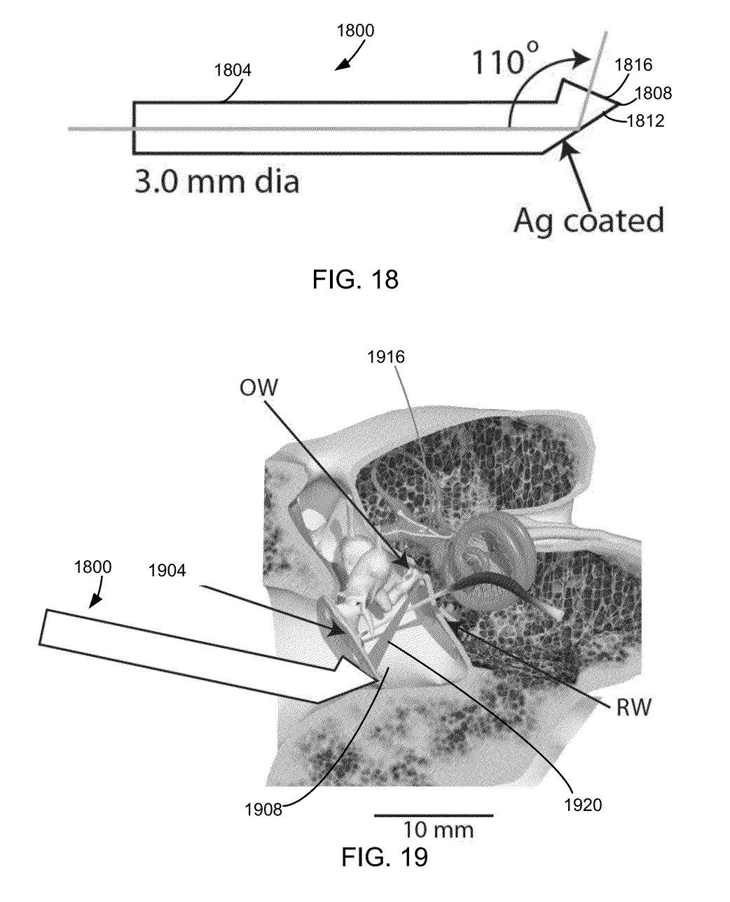

FIG. 18 is a schematic view of another endoscope 1800 used in another embodiment of the invention. In this embodiment the endoscope 1800 comprises a wave guide 1804 and an end piece 1808, where the wave guide 1804 and end piece 1808 are formed from a single piece of material to remove any interface between the wave guide and end piece. The end piece 1808 has an angled and silver coated side 1812 to bend light travelling down the wave guide 1804 at 110°. An end window 1816 has a surface that is close enough to perpendicular to light reflected from the angled and silver coated side 1812 to avoid total internal reflection. In this embodiment, the wave guide 1804 is a GRIN lens and has a diameter of 3.0 mm. The diameter of the wave guide 1804 may be larger than the diameter of a waveguide that is inserted through the tympanic membrane, since the diameter is not constrained by the incision. However, the wave guide must be thin enough to pass through the ear canal.

FIG. 19 is a schematic view of the endoscope 1800 in use in an ear. The end window 1816 of the endoscope is place near but does not pass through the tympanic membrane 1904. Light 1920 is transmitted through the end window 1816 and through the tympanic membrane 1904 and the tympanic chamber 1908 to the round window membrane or oval window footplate of the inner ear 1916, where the light is focused to scan and generate an image of the inner ear 1916. In this embodiment, the image plane where the light is focused is 8 mm from the end window 1816. In other configurations where the endoscope remains in the ear canal and does not pass through the tympanic membrane, the focal plane is between 4 mm and 16 mm from the end window. The end piece bends the light at an angle between 0° and 120°. More preferably, the end piece bends the light at an angle of between 0° to 45°. The exact angle to be selected will depend upon the approach angle through the ear canal, the position of tip of the endoscope relative to the tympanic membrane, and the region of the inner ear to be imaged. All of these factors can vary because of patient-specific factors and their disease. For example, scar tissue within the ear drum may limit the positioning of endoscope to certain regions. In such embodiments the wave guide has a diameter of no more than 4 mm.

Another embodiment of the device is not shown. In this case, the endoscope is used during a trans-mastoid surgical approach to the middle ear. With this approach, an incision is made behind the ear, the mastoid air cells are drilled away, and the oval and round windows of the inner ear are viewed through the facial recess. In this case the angle of the endoscope may vary between 0 to 170°, depending upon the patient's anatomy and the portion of the inner ear that is to be imaged.

An embodiment of the invention allows the examination of soft tissue and other features of the inner ear with high resolution. Such an examination of the vestibular system of the inner ear provides a diagnostic for various types of vertigo. Such an examination of the cochlea of the inner ear provides a diagnostic for various types of hearing loss. An embodiment of the invention allows for the examination of the inner ear for response to sound. An embodiment of the invention also allows for the examination of the response of the inner ear to one or more frequencies of sound. These features provide an additional diagnostic for hearing loss. Since sound may also cause the vibration of the vestibular system, measuring response of the inner ear to sound may also provide a diagnostic for various types of vertigo.

To allow the end of the endoscope to approach the inner ear, the endoscope must be very thin to allow passage through the tympanic membrane or temporal bone or just to the end of the ear canal. Such thin endoscopes have a small field of view (FOV). Since the inner ear is so small, the small FOV is not a limitation for imaging the inner ear. In addition, providing a proper angle to clearly image the inner ear may also be difficult. By providing an end piece that bends the light, the right end piece will bend the light to allow a proper angle for imaging the inner ear. An embodiment of the invention provides a number of interchangeable grin lenses with different end pieces. The right grin lens and mounted end piece is then selected depending on the desired viewing angle and then mounted in the endoscope.

While this invention has been described in terms of several preferred embodiments, there are alterations, permutations, modifications and various substitute equivalents, which fall within the scope of this invention. It should also be noted that there are many alternative ways of implementing the methods and apparatuses of the present invention. It is therefore intended that the following appended claims be interpreted as including all such alterations, permutations, modifications, and various substitute equivalents as fall within the true spirit and scope of the present invention.

What is claimed is:

1. An apparatus, for examining an inner ear, comprising:
an endoscope, comprising:
a wave guide; and
an end piece comprising an end window to be placed a first distance from an inner ear, wherein the waveguide focuses light to create a focal plane the first distance from the end window; and
an optical coherence tomography (OCT) system connected to a second end of the wave guide and comprising an imaging system connected to the OCT system for generating an image of the inner ear.

2. The apparatus, as recited in claim 1, wherein the waveguide and end piece have a diameter no greater than 4 mm.

3. The apparatus, as recited in claim 2, wherein the end piece further comprises a reflector surface.

4. The apparatus, as recited in claim 3, wherein the first distance is between 4 mm and 16 mm.

5. The apparatus, as recited in claim 4, wherein the reflector is a silver coated surface.

6. The apparatus, as recited in claim 5, wherein the wave guide is a gradient index (GRIN) lens.

7. The apparatus, as recited in claim 6, further comprising a sound system comprising:
a sound generator for generating a sound with at least one frequency; and
a controller for controlling the at least one frequency; and
an output device for outputting the at least one frequency.

8. The apparatus, as recited in claim 6, wherein the wave guide has a diameter of no more than 2 mm and wherein the first distance is between 0 mm and 8 mm.

9. The apparatus, as recited in claim 3, wherein the reflector is a silver coated surface.

10. The apparatus, as recited in claim 1, wherein the first distance is between 4 mm and 16 mm.

11. The apparatus, as recited in claim 1, wherein the wave guide is a gradient index (GRIN) lens.

12. The apparatus, as recited in claim 1, further comprising a sound system comprising:
a sound generator for generating a sound with at least one frequency; and
a controller for controlling the at least one frequency; and
an output device for outputting the at least one frequency.

13. The apparatus, as recited in claim 1, wherein the wave guide has a diameter of no more than 2 mm and wherein the first distance is between 0 mm and 8 mm.

14. A computer implemented method for examining the inner ear, comprising:
performing spectral domain optical coherence tomography on the inner ear, comprising;
providing a light beam to the inner ear;
receiving light reflected from the inner ear; and
generating an image of the inner ear from the received light using an optical coherence tomography (OCT) system.

15. The computer implemented method, as recited in claim 14, further comprising:
providing at least one sound signal to the inner ear; and
measuring a vibrational response of the inner ear to the sound signal.

16. The computer implemented method, as recited in claim 15, wherein at least one frequency of the at least one sound signal is known, wherein the measuring the vibrational response is adjusted to the at least one frequency.

17. The computer implemented method, as recited in claim 15, wherein the providing a light beam to the inner ear, comprises:
placing a first end of an endoscope into an ear canal or through a temporal bone, so that the first end is a first distance away from the inner ear, wherein a second end of the endoscope is connected to the OCT system;
focusing light from the endoscope to a focal plane the first distance away from the first end of the endoscope; and
scanning the light on the inner ear.

18. The computer implemented method, as recited in claim 17, wherein the first distance is between 0 mm and 16 mm.

19. The computer implemented method, as recited in claim 17, wherein the placing the first end of the endoscope, further comprises placing the first end through an incision in a tympanic membrane, and wherein the first distance is between 0 mm and 8 mm.

20. An apparatus for examining a surgically exposed inner ear, comprising:
a microscope;
an optical coherence tomography (OCT) system connected to the microscope;
a sound system connected to the OCT system, comprising:
a sound generator for generating a sound with at least one frequency;
a controller for controlling the at least one frequency; and
an output device for outputting the at least one frequency to the OCT system.
